(19) United States
(12) Patent Application Publication
(10) Pub. No.: US 2008/0112891 A1
(43) Pub. Date: May 15, 2008

(54) Encapsulated (Chelate or Ligand) Dendritic Polymers

(75) Inventors: Donald A. Tomalia, Midland, MI (US); Baohua Huang, Mt. Pleasant, MI (US)

Correspondence Address: TECHNOLOGY LAW, PLLC, 3595 N. SUNSET WAY, SANFORD, MI, 48657, US

(73) Assignee: Dendritic Nanotechnologies, Inc., Mt. Pleasant, MI (US)

(21) Appl. No.: 11/795,091

(22) PCT Filed: Feb. 15, 2006
(86) PCT No.: PCT/US06/05334

§ 371 Date: Jul. 10, 2007

Related U.S. Application Data
(60) Provisional application No. 60/652,991, filed on Feb. 15, 2005.

(51) Int. Cl. A61K 49/10; A61K 31/787; C08G 63/91; C08G 63/48

(52) U.S. Cl. 424/9.322; 424/78.27; 525/437; 525/54.1; 525/54.2

(57) ABSTRACT

An encapsulated chelate dendritic polymer and an encapsulated ligand dendritic polymer are disclosed which have unique properties. These encapsulated chelate dendritic polymers may have associated with its dendritic polymer surface target directors, proteins, DNA, RNA (including single strands) or any other moieties that will assist in diagnosis, therapy or delivery of this encapsulated chelate dendritic polymer. These encapsulated dendritic polymers are suitable as contrast agents for use in imaging in an animal, for other imaging techniques, for EPR, and as scavenger agents for chelant therapy. Formulations for these uses are also included within the scope of this invention.

### STATEMENT REGARDING FEDERALLY SPONSORED RESEARCH OR DEVELOPMENT

This application was funded by grants from the US Army Research Laboratory under agreement numbers DAAD19-03-2-0012 and W911NF-04-2-0030 to Central Michigan University, which subcontracted to Dendritic Nanotechnologies, Inc. The US Government has certain rights to this application for its use in accord with the terms of those grants and agreement.

### FIELD OF THE INVENTION

The present invention concerns the use of dendritic polymers as carriers for magnetic resonance imaging (MRI) contrast agents wherein the contrast agent is a chelate (a metal complexed to a ligand) that must be encapsulated within the interior of the dendritic polymer. Additionally, the chelate (i.e., metal+ligand; that can be a contrast agent) may also be associated with the surface of the dendritic polymer in addition to being encapsulated. Other desirable moieties may be associated with the dendritic polymer surface such as target directors, proteins, DNA, RNA (including single strands) or any other moieties that will assist in diagnosis, therapy or delivery of this encapsulated chelate dendritic polymer. These encapsulated chelate dendritic polymers because of their controlled nanoscale sizes may manifest MRI blood pool imaging characteristics or be used as size specific targeting for imaging primary cancer tumors or other highly vascularized in vivo domains by techniques referred to as enhanced permeability and retention (EPR). Additionally, the dendritic polymer with the ligand encapsulated may be used in a variety of ways, wherein the desired metal reagents for MRI imaging or other metal containing reagents useful for computerized tomography (i.e., CT scans), diagnostic or radioactive reagents, or heavy metal such as gold for other imaging techniques, may be added later. These encapsulated chelate dendritic polymers may also be use to image plants, for example to determine the pathway or movement of various chemicals and nutrients through the plant. Alternatively, the encapsulated ligand (or chelating agent) dendritic polymer may be used as scavengers to absorb an unwanted or excess of a metal from the body, such as in “chelation therapy”.

### BACKGROUND OF THE INVENTION

MRI is a non-invasive diagnostic technique which produces well resolved cross-sectional images of soft tissue within an animal body, preferably a mammalian animal body, more preferably a human body. This technique is based upon the property of certain atomic nuclei (e.g. water protons) which possess a magnetic moment [as defined by mathematical equations; see G. M. Barrow, Physical Chemistry 3rd Ed., McGraw-Hill, N.Y. (1973)] to align in an applied magnetic field. This technique has proven to be so important that Dr. Paul Lauterbur, the inventor, was awarded the Nobel Prize in 2003.

Once aligned, this equilibrium state can be perturbed by applying an external radio frequency (RF) pulse which causes the protons to be tilted out of alignment with the magnetic field. When the RF pulse is terminated, the nuclei return to their equilibrium state and the time required for this to occur is known as the relaxation time. The relaxation time consists of two parameters known as spin-lattice (T₁) and spin-spin (T₂) relaxation and it is these relaxation measurements which give information on the degree of molecular organization and interaction of protons with the surrounding environment.

Since the water content of living tissue is substantial and variations in content and environment exist among tissue types, diagnostic images of biological organisms are obtained which reflect proton density and relaxation times. The greater the differences in relaxation times (T₁ and T₂) of protons present in tissue being examined, the greater will be the contrast in the obtained image [J. Magnetic Resonance 33, 83-106 (1979)].

It is known that paramagnetic chelates possessing a symmetric electronic ground state can dramatically affect the T₁ and T₂ relaxation rates of juxtaposed water protons and that the effectiveness of the chelate in this regard is related, in part, to the number of unpaired electrons producing the magnetic moment [Magnetic Resonance Annual, 23-266, Raven Press, N.Y. (1985)]. It has also been shown that when a paramagnetic chelate of this type is administered to a living animal, its effect on the T₁ and T₂ of various tissues can be directly observed in the magnetic resonance (MR) images with increased contrast being observed in the areas of chelate localization. It has therefore been proposed that stable, non-toxic paramagnetic chelates be administered to animals in order to increase the diagnostic information obtained by MRI [Frontiers of Biol. Energetics I, 752-759 (1978); J. Nucl. Med. 25, 506-513 (1984); Proc. of NMR Imaging Symp. (Oct. 26-27, 1980); F. A. Cotton et al., Adv. Inorg. Chem. 634-639 (1966)]. Paramagnetic metal chelates used in this manner are referred to as contrast enhancement agents or contrast agents.

At the present time, the only commercial contrast agents available in the United States of America are: the complex of gadolinium with diethylenetriaminepentaacetic acid [DTPA-Gd⁺³-Magnevist™ by Schering AG, extracellular for central nervous system (CNS) and whole body]; a DO3A derivative [1,4,7-tris(carboxymethyl)-10-(2-hydroxypropyl)-1,4,7,10-tetraazacyclododecanato]-gadolinium (ProHance™ by Squibb, extracellular for whole body and some CNS); [1,4,7-tris(carboxymethyl)-10-(1,2,3-trihydroxypropyl)-1,4,7,10-tetraazacyclododecanato]-gadolinium (Gadovist™ by Schering AG, extracellular agent for CNS indications, e.g. lesions in the brain); aqua[5,8-bis(carboxymethyl)-11[2-(methylamino)-2-oxoethyl]-3-oxo-2,5,8,11-tetraazatridecan-13-oato(3-)-N⁵,N⁶,N¹¹,O³,O⁵,O⁸,O¹¹,O¹³]gadolinium hydrate (Omniscan™ by Amersham Health, for CNS and cardiovascular disease ); mangafodipir (Telescan™ by Amersham Health, an intravenous agent for liver lesions); gadobenate dimeglumine (MultiHance™ by Bracco, for liver lesions and brain lesions); manganese chloride tetrahydrate (LumenHance™ by ImaRx); [8,11-bis(carboxymethyl)-14-{2-[(2-methoxyethyl)amino]-2-oxoethyl}-6-oxo-2-oxa-5,8,11,14-tetraazahexadecan-16-oato(3-)]gadolinium (OptiMARK™ by Mallinckrodt, for brain, spine and liver); poly[N-(2-aminoethyl)-3-aminopropyl]siloxane-coated non-stoichiometric magnetic FeO (GastroMARK™ by Mallinckrodt, oral agent for gastrointestinal lesions); iron oxide nanoparticles (Combidex™ by Advanced Magnetics, Inc., for lymph nodes); aqueous colloid of superparamagnetic iron oxide (Feridex™ by Advanced Magnetics Inc., for liver lesions) and silicone coated superparamagnetic iron oxide (GastroMARK™ by Advanced Magnetics Inc., for loops in the bowel). Magnevist™ and ProHance™ are each considered as a non-specific/perfusion agent since it freely distributes in extracellular fluid followed by efficient elimination through the renal system. Magnevist™ has proven to be extremely valuable in the diagnosis of brain lesions since the accompanying breakdown of the blood/brain barrier allows perfusion of the contrast agent into the affected regions. In addition to Magnevist™, Guerbet is commercially marketing a macrocyclic perfusion agent (Dotarem™) which presently is only available in Europe. ProHance™ is shown to have fewer side effects than Magnevist™. A number of other potential contrast agents are in various stages of development.

Although dendritic polymers have been used as carriers of contrast agents (see U.S. Pat. Nos. 5,527,524; 5,364,614; 5,820,849; 6,054,117; 6,063,361; 5,650,136; 6,183,724; and 5,911,971; and WO2003/001218; and WO2004/019998), these prior dendritic carriers have not encapsulated the desired metal in the interior of the dendritic polymer by use of a chelating agent or ligand. Thus these prior systems used only the metal encapsulated within the interior of the dendritic polymer.

Enhanced permeability and retention (EPR) is a method for a passive targeting usually of tumors where the size of the particle used is important. The purpose is to obtain selectively concentrated particles in the tumor vasculature for imaging or viewing and the size for elimination from the body thereafter. [See Nature Reviews 3, 347-360 (2003).]

The use of various chelating systems as scavengers for heavy metal poisoning is known. Such scavengers are used in chelation therapy for the removal of various undesired metals or excess presence of metal. On example is in lead poisoning where lead is removed with the use of a ligand, ethylenediaminetetraacetic acid (EDTA), by injection of the ligand into the blood stream and allowing the chelate (ligand-lead) to be excreted through the kidneys. [See A Textbook on EDTA Chelation Therapy, ed. E. M. Cranton, M.D., 2d ed., pub. 2001.] Another example is the removal of plutonium from the body after such toxic exposure using the ligand, diethylene-triaminepentaacetic acid (DTPA) or hydroxypyridinone. [See J. Med. Chem. 45, 3963-3971 (2002).] Other such metal removal for mercury and heavy metals have been performed using the ligand dimercaptosuccinic acid (DMSA; Chemet) or dimercaptopropane sulfonic acid (DMPS; Dimaval). [See Chem. Res. Toxicol. 17, 999-1006 (2004).] Severe iron overload, known as haemochromatosis, has used as chelators desferrioxamine, hydroxypyridones, and pyridoxal hydrazones, although they have known disadvantages. [See J. Med. Chem. 45, 5776-5785 (2002); Semin. Hematol. 32, 304-312 (1995); Blood 89, 739-761 (1997).]

None of these prior scavengers have encapsulated the desired metal in the interior of a dendritic polymer by use of a chelating agent.

### SUMMARY OF THE INVENTION

In its broadest aspect, the present invention is directed to an encapsulated chelate dendritic polymer. These encapsulated chelate dendritic polymers are suitable as MRI or computerized tomography (CT) contrast agents for use in imaging an animal or plant, and therapeutic agents when a radioactive metal is used in the chelate. Additionally, the present invention is directed to an encapsulated ligand dendritic polymer for use as a scavenger for metals and their ionic moieties to remove such metals from the environment, such as arsenic from water systems, toxic presence of metals in tissue of both animals and plants. Formulations for these uses are also included within the scope of this invention.

### BRIEF DESCRIPTION OF THE DRAWINGS

FIG. 1 shows a ladder of ethylenediamine (EDA) core, dendri-PAMAM tris-(OH)_{z} surface dendrimers of G=4, 5 and 6, respectively. Where the Figure shows in lane 2 a naked (no chelate) G=4 dendrimer; and in lane 3 (labeled 1) on the Figure a G=4 dendrimer with DTPA-Gd⁺³ encapsulated; and in lane 4 (labeled 2) is G=5 dendrimer with the chelate of DTPA-Gd⁺³ encapsulated. The gel is a 15% homogenous poly(acrylamide) gel/0.1% sodium dodecyl sulfate (SDS).

FIG. 2 shows an electropherogram (PAGE) on a 4-20% cross-linked poly(acrylamide) gradient gel displaying a ladder of EDA core, dendri-PAMAM, (NH₂)_{z} surfaced dendrimers (i.e., G=0-6). The Figure shows in lane 2 (labeled number 4) a naked (no chelate) G=4; and lanes 3, 4 and 5 (labeled by numbers 1, 2 and 3, respectively) display amine surfaced dendrimers (i.e., G=2, 3, 4) complexed with DTPA-Gd⁺³. Lane 6 (labeled number 5) is the G4 amine surface dendrimer complexed with excess DTPA-Gd⁺³ then adding carbodiimide and is also the PAGE for Example 5, which demonstrates both encapsulation as well as attachment of chelate to the dendrimer surface by its position on the gel.

FIG. 3 shows a depiction of a G=3 and G=4 PAMAM dendrimer with DTPA-Gd⁺³ encapsulated at a ratio of tertiary amines to chelate of 2:1.

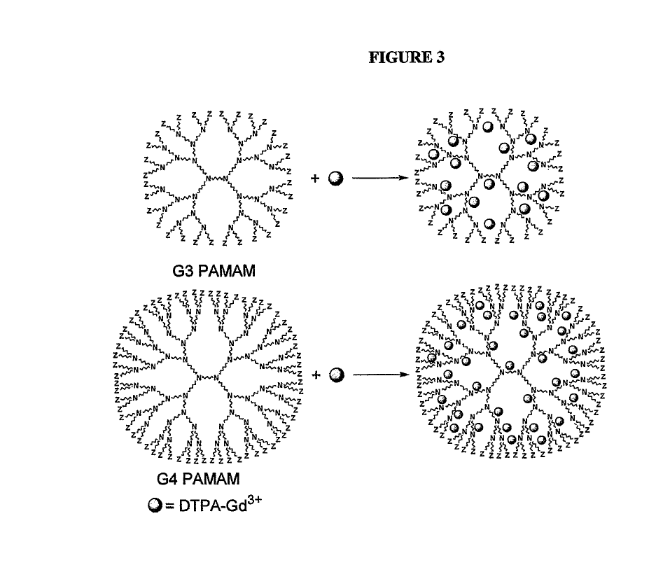

FIG. 4 shows a depiction of a G=4 PAMAM dendrimer with DTPA-Gd⁺³ encapsulated and having the surface functionalized with covalently bound DTPA-Gd⁺³, thereby have the chelate both on the surface and encapsulated.

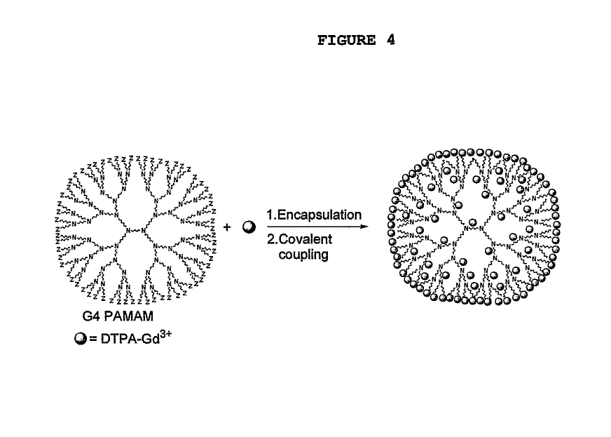

FIG. 5 shows a depiction of a G=4 PAMAM dendrimer with DTPA-Gd⁺³ encapsulated and having the surface functionalized by non-covalent association with DTPA-Gd⁺³, thereby have the chelate both on the surface and encapsulated.

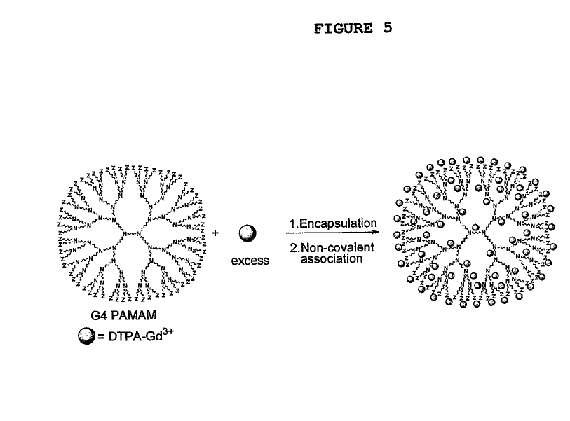

FIG. 6 shows a depiction of a G=4 PAMAM dendrimer with a ligand encapsulated (i.e., an encapsulated ligand dendritic polymer), then later adding a metal to form the encapsulated chelate dendritic polymer.

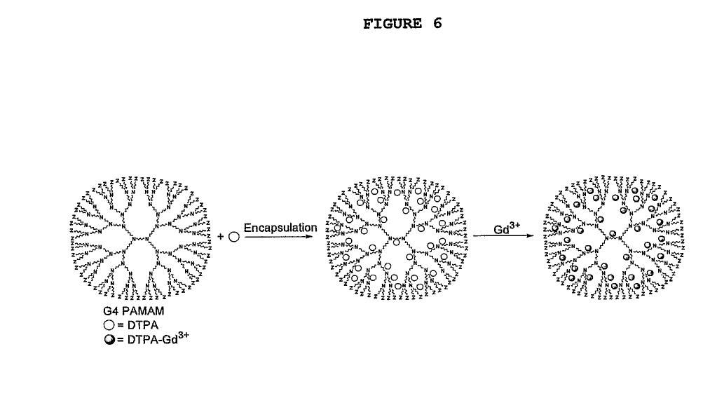

### DETAILED DESCRIPTION OF THE INVENTION

The present invention concerns the use of dendritic polymers as carriers for magnetic resonance imaging (MRI) contrast agents wherein the contrast agent is a chelate (a metal complexed to a ligand) that must be encapsulated within the interior of the dendritic polymer. Additionally, the chelate (i.e., metal+ligand; that can be a contrast agent) may also be associated with the surface of the dendritic polymer in addition to being encapsulated. These encapsulated chelate dendritic polymers have use as pharmaceutical imaging agents, and because of their controlled nanoscale sizes may manifest MRI blood pool imaging characteristics or be used as size specific targeting for imaging primary cancer tumors or other highly vascularized in vivo domains by techniques referred to as enhanced permeability and retention (EPR). Additionally, the dendritic polymer with the ligand encapsulated may be used in a variety of ways, wherein the desired metal reagents for MRI imaging or other metal containing reagents for computerized tomography (i.e., CT scans) diagnostic radioactive reagents or heavy metal such as gold for other imaging techniques may be added later. These encapsulated chelate dendritic polymers may also be use to image plants, for example to determine the pathway or movement of various chemicals and nutrients through the plant. Alternatively, the encapsulated ligand (or chelating agent) dendritic polymer may be used as scavengers to absorb an unwanted or excess of a metal from the body, such as in “chelation therapy”.

In its broadest aspect, the present invention is directed to dendritic polymers having encapsulated within its interior a chelate. The chelate is also termed a complex and comprises a chelating agent or ligand and a metal. This encapsulated chelate dendritic polymer is used as a contrast agent for imaging in animals, preferably mammals, especially humans, and plants. Also these encapsulated chelate dendritic polymers may be used as therapeutic agents when a radioactive metal is used in the complex. These encapsulated chelate dendritic polymers may also be used for enhanced permeability and retention (EPR) studies because of their controlled size at the nanoscale level. The need for such encapsulation by the chelate within the interior of the dendritic polymer, where the metal is chelated to a chelating agent or ligand, overcomes many disadvantages of these prior carrier systems.

Additionally, these dendritic polymers when they are encapsulating a ligand may be used as scavengers to remove various metals, such as from the environment, for example arsenic from water systems for purification and other metal contamination areas, but especially from an animal body in vivo.

The term “metal” or “metal reagent” as used herein means any element on the periodic table that is usually considered a metal or psudometal in all its forms (e.g., zero valence state, radioactive, non-radioactive) and includes any suitable counter ions when the metal is ionic. These metals may be used for diagnostic or therapeutic purposes in an animal or plant; or considered desirable to be removed from the environment; or toxic to animals or plants and therefore wanting to be removed from the environment or from an animal or plant. Such metals may also be a part of chelation therapy.

There are a number of metal ions which can be considered when undertaking the design of an MRI contrast agent. A “paramagnetic nuclide” of this invention means a metal ion which displays spin angular momentum and/or orbital angular momentum. The two types of momentum combine to give the observed paramagnetic moment in a manner that depends largely on the atoms bearing the unpaired electron and, to a lesser extent, upon the environment of such atoms.

The metals which can be used in these encapsulated chelate dendritic polymers include paramagnetic or magnetic metals, such as metals in the Periodic Table Groups VIIIA (Fe, Co, Ni, Ru, Rh, Pd, Os, Ir, Pt), IVB (Pb, Sn, Ge), IIIA (Sc, Y, lanthanides and actinides), IIIB (B, Al, Ga, In, Tl), IA (Li, Na, K, Rb, Cs, Fr), and IIA (Be, Mg, Ca, Sr, Ba, Ra). For other uses detailed herein these metals can be radioactive and used for diagnosis or therapy. The above Groups are designated using the IUPAC form of nomenclature.

In practice, however, the most useful paramagnetic metal ions for MRI are gadolinium (Gd⁺³), iron (Fe⁺³), manganese (Mn⁺²) and (Mn⁺³), and chromium (Cr⁺³), because these ions exert the greatest effect on water protons by virtue of their large magnetic moments. In a non-complexed form (e.g. GdCl₃), these metal ions are toxic to an animal or plant, thereby precluding their use in the simple salt form. Therefore, a fundamental role of the organic chelating agent (also referred to as a ligand) is to render the paramagnetic metal non-toxic to the animal or plant while preserving its desirable influence on T₁ and T₂ relaxation rates of the surrounding water protons. Especially preferred are Fe⁺³, Gd⁺³, Mn⁺² and Mn⁺³ which are available commercially, e.g. from Aldrich Chemical Company. The anion present is halide, preferably chloride, or salt free (metal oxide).

For other uses intended for these encapsulated chelate dendritic polymers, the metal may be selected for desired imaging application. Alternatively, for the removal of a noxious metal reagent, the dendritic polymer has the ligand encapsulated within the dendritic polymer structure which is then a scavenger agent. In this case the dendritic polymer with the ligand encapsulated can then remove undesirable metal reagents, including radioactive isotopes, from the environment or from an animal or plant. Some examples of such metal reagents are lead, arsenic, cadmium, plutonium, uranium, technetium, platinum, iron, calcium, mercury, gold and other heavy metals and heavy metal salts possessing a variety of counter ions. [For a review of these uses see Saul Green, Chelation Therapy: Unproven Claims and Unsound Theories, Quickwatch Home Page, revised Mar. 28, 2002.]

Suitable chelating agents or ligands that may be used are any that will bind to the desired metal reagents and enter the interior of a dendritic polymer as a pre-formed chelate or complex. Alternatively, this invention includes any chelating agent or ligand that will enter the dendritic polymer independently or in combination with the metal to produce the desired chelate within the dendritic polymer interior. Aminocarboxylic acid chelating agents have been known and studied for many years. Many chelates are known where the ligand and metal associate in a manner conducive for the use of the chelate. [See for example Chemistry of the Metal Chelate Compounds, by Arthur Earl Martell, pub. Prentice-Hall; and Chem. Rev. 99, 2293-2352 (1999).] Typical of the classes of such ligands are the linear organic acids, macrocyclics, macrocyclic derivatives, kryptates, phosphines, thioalkyl, ethers, carboxylates, thioureas, phosphonic acids, methylenephosphonic acids, sulfonic acids, and macrocyclic polypeptides. Especially useful as ligands are aminocarboxylic acids are nitrilotriacetic acid (NTA), ethylenediaminetetraacetic acid (EDTA), hydroxyethylethylenediaminetriacetic acid (HEDTA), diethylenetriaminepentaacetic acid (DTPA), trans-1,2-diaminocyclohexanetetraacetic acid (CDTA). and 1,4,7,10-tetraazacyclododecanetetraacetic acid (DOTA), 1,4,7,10-tetraazacyclododecane-1,4,7-triacetic acid (DO3A), and 2-(p-isothiocyanatobenzyl)-6-methyl-diethylene-triaminepentaacetic acid (IB4M). Other chelating agents that have now been suggested include hydroxypyridinone (HOPO) and TREN-1-methyl-3,2-HOPO [Bioconjugate Chem. 16, 3-8 (2005)].

Numerous bifunctional chelating agents based on aminocarboxylic acids have been proposed and prepared. For example the cyclic dianhydride of DTPA [Hnatowich et al. Science 220, 613-615, (1983); U.S. Pat. No. 4,479,930] and mixed carboxycarbonic anhydrides of DTPA [Gansow, U.S. Pat. Nos. 4,454,106 and 4,472,509; Krejcarek et al., Biochem. and Biophys. Res. Comm. 77, 581-585, (1977)] have been reported. When the anhydrides are coupled to proteins the coupling proceeds via formation of an amide bond thus leaving four of the original five carboxymethyl groups on the diethylenetriamine (DETA) backbone [Hnatowich et al., Int. J. Appl. Isot. 33, 327-332, (1982)]. These chelating agents are most useful on the surface of the encapsulated chelate dendritic polymer.

In addition, U.S. Pat. Nos. 4,432,907 and 4,352,751 disclose bifunctional chelating agents useful for binding metal ions to “organic species such as organic target molecules or antibodies.” As in the above for the dendritic polymer surface, coupling is generally obtained via an amide group through the utilization of diaminotetraacetic acid dianhydrides. Examples of anhydrides include dianhydrides of EDTA, CDTA, propylenediaminetetraacetic acid and phenylene 1,2-diaminetetraacetic acid. U.S. Pat. No. 4,647,447 discloses several complex salts formed from the anion of a complexing acid for use in various diagnostic techniques. Conjugation via a carboxyl group of the complexing acid is taught which gives a linkage through an amide bond. Alternatively, a variety of other bioconjugation methods widely recognized by those skilled in the art may be used to covalently attach appropriate metal chelates, targeting groups or desired biocompatibility/distribution groups to the dendritic polymer surface of the encapsulated chelate dendritic polymer. [See G. Hermanson, Bioconjugate Techniques, Academic Press, London (1996).]

Of course, all of the ligands mentioned above that serve as commercial ligands in contrast agents, EPR or as scavengers may also be used in the present invention as a ligand.

The complexes of the metal and chelating agent are generally at a ligand to metal molar ratio determined by the stiochimetry of the ligand. The metal to ligand molar ratio is generally from about 1:1 to about 3:1, more preferably from about 1:1 to about 1.5:1. Preferably, the molar ratio of metal to binding sites on the ligand is about 1:1.

The chelate to dendrimer molar ratios are at least about 1:1, preferably from about 1:1 to a value that is determined by the amount of void space present in the interior of the dendritic polymer. A large excess of ligand is usually undesirable since uncomplexed ligand present and not in the encapsulated chelate dendritic polymer interior may be toxic to the animal or may result in cardiac arrest or hypocalcemic convulsions. Thus after the metal-chelating agent complex is formed, excess ligand is preferably removed prior to encapsulating within the dendritic polymer or after the encapsulated chelate dendritic polymer is formed. Because excess free metal in the body is also toxic, excess metal is also undesirable. This metal-ligand complex may be understood to be a “guest molecule” within the dendritic polymer which serves as the “host molecule”.

When the ligand only is encapsulated within the dendritic polymer for use as a scavenger system, then the ligand may be understood to be a “guest molecule” for the dendritic “host molecule”. The metal is entrapped by the dendritic polymer encapsulated ligand during use and forms the encapsulated chelate with the dendritic polymer. The encapsulated chelate dendritic polymer can be understood to be a complex of metal and ligand, encapsulated within the dendritic polymer to form a three component conjugate system comprising a metal, ligand and dendritic polymer.

When the chelate or ligand is encapsulated within the dendritic polymer the nature of the bonding between the interior of the dendrimer and the ligand or chelate is best described as being associated with each other. The term “associated with” includes an attachment or linkage by means of covalent bonding, hydrogen bonding, adsorption, absorption, metallic bonding, van der Waals forces, ionic bonding, or coulombic, hydrophobic, hydrophilic, or chelation forces, or any combination thereof. Thus the term “encapsulation” or “encapsulated” as used herein for the ligand or chelate within a dendritic polymer means entrapment or associated with (as defined above) of the ligand or chelate with the interior of the dendritic polymer. The metal must be associated with the ligand such that at least two arms or binding sites of the ligand are associated with the metal. These two sites may be on the same ligand or on multiple ligands.

The dendritic polymers that can be used in the encapsulated chelate dendritic polymers are well known to those skilled in this art. Some examples of dendritic polymers include but are not limited to such polymers as: random hyperbranched polymers (see for example the polylysine polymers in U.S. Pat. Nos. 4,360,646 and 4,410,688); dendrimers (provided that there is an interior void space for the chelate) (see for example U.S. Pat. Nos. 4,507,466, 4,558,120, 4,587,329 and 4,568,737); dendrigraft polymers, dendrons, dendritic megamers, linear-dendritic architectural copolymers, cross-linked (bridged) dendritic polymers (see for example U.S. Pat. No. 4,737,550) and hypercomb-branched polymers (see for example U.S. Pat. No. 5,631,329); non-crosslinked polybranched polymers (see for example U.S. Pat. No. 5,773,527); star comb branched polymers (see for example U.S. Pat. No. 4,690,985); convergent self-branching polymers (see for example WO 98/36001); and core-shell tecto(dendrimers) (see for example U.S. Pat. No. 6,635,720), to mention a few. This includes dendritic compositional copolymers (see for example U.S. Pat. Nos. 5,739,218; 5,902,863). This includes dendritic polymers synthesized by convergent, divergent or self assembly type methods (see J. M. Frechet and D. A. Tomalia, Dendrimers and Other Dendritic Polymers, pub. J. Wiley (2001). Especially preferred dendritic polymers are those which are dendrimers. Such dendrimers are defined as unimolecular assemblages that posses three distinguishing architectural features, namely, (a) an initiator core, (b) interior layers (generations, G) composed of repeating units, radially attached to the initiator core, and (c) an exterior surface of terminal functionality (surface groups, Z). Such dendrimers include but are not limited to polyamidoamine (PAMAM) dendrimers, poly(propyleneimine) (PPI) dendrimers, poly(triazine)dendrimers, poly(ether-hydroxylamine) (PEHAM) dendrimers, which may have their Z groups modified or selected to force the chelating agents exclusively into the dendritic polymer interior or in combination with encapsulation, allow association with the surface of the dendritic polymer. Examples of some such Z surfaces are those which do not interact with the ligand; such Z groups are hydroxyl, ester, acid, ether, carboxylic salts, alkyls, glycols, such as for example hydroxyl groups especially those from amidoethanol, amidoethylethanolamine, tris(hydroxymethyl)amine, carbomethoxypyrrolidinone, amido, thiourea, urea, carboxylate, succinamic acid and polyethylene glycol or primary or primary, secondary or tertiary amine groups with or without hydroxyl alkyl modifications. Other suitable surface groups may include any such functionality that would allow associative attachment (associate with) the dendritic polymer surface and include but are not limited to receptor mediated targeting groups (e.g., folic acid, antibodies, antibody fragments, single chain antibodies, proteins, peptides, oligomers, oligopeptides, or genetic materials) or other functionality that would facilitate biocompatibility, biodistribution, solubility or modulate toxicity.

The ratio of chelate to tertiary amino groups located in the interior of the PAMAM dendrimers described by the later examples and teachings that the chelate is residing in the interior rather than on the surface, regardless of the surface groups Z. Any interior group capable of associating with the ligand is suitable. However, the exclusive or substantial presence of the chelate only on the interior of the dendritic polymer can be assured only if the surface groups have been designed or modified to be non-reactive or no associative with the ligand or chelate. Such Z groups which permit this result include any non-basic functionality such as hydroxyl groups from tris(hydroxylmethyl)amides, amidoalkanol, and thioalkanol moieties, amido, amidoalkyl, urea, thiourea, ether, thioether moieties, or moieties such as esters, carboxylic acid, sulfonic acid or polyethylene glycol (PEG) groups. To ensure that the chelate resides in the interior, the surface should not contain groups that may form covalent, charge neutralization or association/complex type connectivity. It is preferred that the chelate reside in the interior of the dendritic polymer because of the stability and reduced toxicity of this functionalized delivery system as well as the controlled nano-scale sizes of the dendritic polymers which may be defined by the generation size of the dendrimer or from about 1 kD to about 60 kD or about 1 nm to about 11 nm. At these nano-scale sizes (which exceed the size of Magnivist™), these encapsulated chelate dendritic polymers manifest “blood pool agent” properties that are normally associated with macromolecular conjugates. This feature minimizes the leakage of contrast agent from the blood vessels into the interstitial space while masking any toxicity of the metal or chelate. Also, these encapsulated chelate dendritic polymers exhibit enhanced solubilities and may aid in the control of solubility of the chelate when encapsulated in the dendritic polymer. When the encapsulated chelate dendritic polymer is repeatedly dialyzed (as further shown in the examples below), the ratio of chelate to interior tertiary amines remains consistent as shown by gravimetric weight gain which was consistent with mass spectrometry analysis. The dimensions of the encapsulated chelate dendritic polymer as determined by PAGE remains at the dimensions expected unless further surface groups are later attached. Even after repeated dialysis of an encapsulated chelate dendritic polymer, in water no substantial loss of chelate or metal was detected. In the case of the PAMAM dendrimers, the chelate binds by association with the tertiary amines in the interior, and also secondary and primary amines when present, to form a stable encapsulated chelate even after repeated dialysis. This permits a higher loading of the chelate into the interior, ranging from a 1:1 molar ratio of chelate to the tertiary amines in the dendritic polymer to an upper ratio which is determined by the available void space for chelate residency within the dendritic polymer.

In another embodiment of the present invention, the encapsulated chelate dendritic polymer may have the chelated metal present on both in the interior and on the surface of the encapsulated chelate dendritic polymer. For this class of encapsulated chelate dendritic polymers, the surface groups Z are typically basic, acidic, or possess the ability to either charge neutralize or molecularly complex with functionality present on the chelate or ligand. Some preferred functionalities include primary, secondary or tertiary amines and their hydroxylalkaylated analogues.

Although prior dendritic polymers have served as carriers for various materials, there are none which have had a chelated metal carried or encapsulated within the dendritic polymer interior in the manner of this invention. In a preferred method the metal is complexed to the chelating agent and the resulting chelate is associated within the dendritic polymer. The advantage of having this chelate encapsulated within the dendritic polymer is loading, toxicity, uniformity of size for reduction of leakage into tissue, solubility, stability, biocompatibility, and these properties aid clearance through the body, and effective life of the imaging or scavenging agent in the body.

Having a controlled size of the encapsulated chelate dendritic polymer and the encapsulated ligand dendritic polymer provides improvements over other systems. For diagnostic biomedical imaging and for enhanced permeability and retention (EPR) delivery of diagnostic/therapeutic agents to cancer tumors or other tumors these encapsulated dendritic polymers provide controlled nanoscale sizes and a systematic protocol for effective administration that was not possible before. This property for EPR was discussed by Ruth Duncan in Nature Reviews, Drug Discovery, 3, 347-360 (2003). Additionally, the impact of designing controlled sizes of nanoscale dimensions into these encapsulated chelate or ligand dendritic polymers as contrast agents or scavenger agents may be effectively used to enhance and control their in vivo elimination routes and their physical characteristics as blood pool agents as well as impact their biodistribution to targeted tissue, organs, or disease sites [see for example Molecular Imaging 2(1), 1-10 (January 2003)].

As used herein, “pharmaceutically-acceptable salts” means any salt or mixtures of salts of an encapsulated chelate dendritic polymer which is sufficiently non-toxic to be useful in therapy or diagnosis of animals, preferably mammals, more preferably humans. Thus, the salts are useful in accordance with this invention. Representative of those salts formed by standard reactions from both organic and inorganic sources include, for example, sulfuric, hydrochloric, phosphoric, acetic, succinic, citric, lactic, ascorbic, maleic, fumaric, palmitic, cholic, palmoic, mucic, glutamic, gluconic acid, d-camphoric, glutaric, glycolic, phthalic, tartaric, formic, lauric, steric, salicylic, methanesulfonic, benzenesulfonic, sorbic, picric, benzoic, cinnamic acids and other pharmaceutically acceptable acids. Also included are salts formed by standard reactions from both organic and inorganic sources such as ammonium or 1-deoxy-1-(methylamino)-D-glucitol, alkali metal ions, alkaline earth metal ions, and other pharmaceutically acceptable ions. Particularly preferred are the salts of the encapsulated chelate dendritic polymer where the salt is potassium, sodium, or ammonium.

The encapsulated chelate dendritic polymer can be prepared in a number of ways. In one embodiment the metal is first chelated to the chelating agent by methods well known in the art. Thus, for example, see Chelating Agents and Metal Chelates, Dwyer & Mellor, Academic Press (1964), Chapter 7. See also methods for making amino acids in Synthetic Production and Utilization of Amino Acids, (edited by Karneko, et al.) John Wiley & Sons (1974). An example of the preparation of a complex involves reacting diethylenetriaminepentaacetic acid (DTPA) with the metal ion under aqueous conditions at a pH from 5 to 7. The complex formed is by a chemical bond and results in a stable paramagnetic nuclide composition, e.g. stable to the disassociation of the paramagnetic nuclide from the ligand.

A process to make the chelate follows well known methods. The ligand (e.g., DTPA) is dissolved in a solvent (e.g., water). A large excess of the specific metal salt (e.g., gadolinium nitrate) is added to the solution. The reaction mixture is stirred a room temperature (about 18-28° C.) for 2-8 hours. The chelate is purified using an ion exchange column followed by removal of solvent to provide the chelate (e.g., DTPA-Gd⁺³), usually as a solid.

When the chelate made above (e.g., DTPA-Gd⁺³) is added to a solution (e.g., water) of desired dendritic polymer (e.g., G4-PAMAM-OH), and stirred at room temperature (about 18-28° C.) for a desired amount of time (e.g., 2-3 days). The desired encapsulated chelated dendritic polymer product forms spontaneously. The product is then purified by dialysis against water followed by removal of the solvent to give the encapsulated chelate dendritic polymer (e.g., encapsulated DTPA-Gd⁺³ PAMAM).

When the ligand (e.g., DTPA) is added to a solution of the desired dendritic polymer (e.g., PAMAM), the mixture is stirred at room temperature (about 18-28° C.) for a desired amount of time (e.g., 2-3 days). The product is then purified by dialysis against water followed by removal of the solvent to give the encapsulated ligand dendritic polymer (e.g., encapsulated DTPA PAMAM).

When the ligand, metal and dendritic polymer are mixed together in one pot process, a dendrimer (e.g., G4-PAMAM-OH), ligand (e.g., DTPA), and metal salt (e.g., gadolinium nitrate) are added to a solvent (e.g., water) and stirred at room temperature (about 18-28° C.) for a desired amount of time (e.g., 2-3 days). The desired encapsulated chelated dendritic polymer product forms spontaneously. The product is then purified by dialysis against water followed by removal of the solvent to give the encapsulated chelate dendritic polymer (e.g., encapsulated DTPA-Gd⁺³ PAMAM).

When the encapsulated ligand dendritic polymer (e.g., encapsulated DTPA PAMAM) is mixed with a metal salt (e.g., gadolinium nitrate) in a solvent (e.g., water) and stirred at room temperature (about 18-28° C.) for a desired amount of time (e.g., 2-8 hours). The desired encapsulated chelated dendritic polymer product forms spontaneously. The product is then purified by dialysis against water followed by removal of the solvent to give the encapsulated chelate dendritic polymer (e.g., encapsulated DTPA-Gd⁺³ PAMAM).

Some preferred examples of chelates that can be used in the interior of a dendritic polymer are gadolinium diethylenetriaminepentaacetic acid (Gd DTPA), [1,4,7-tris(carboxymethyl)-10-(2-hydroxypropyl)-1,4,7,10-tetraazacyclododecanato]-gadolinium (ProHance™); [1,4,7-tris(carboxymethyl)-10-(1,2,3-trihydroxypropyl)-1,4,7,10-tetraazacyclododecanato]-gadolinium (Gadovist™); aqua[5,8-bis(carboxymethyl)-11[2-(methylamino)-2-oxoethyl]-3-oxo-2,5,8,11-tetraazatridecan-13-oato(3-)-N⁵,N⁶,N¹¹,O³,O⁵,O⁸,O¹¹,O¹³]gadolinium hydrate (Omniscan™); mangafodipir (Telescan™); gadobenate dimeglumine (MultiHance™); manganese chloridetetrahydrate (LumenHance™); and [8,11-bis(carboxymethyl)-14-{2-[(2-methoxyethyl)amino]-2-oxoethyl}-6-oxo-2-oxa-5,8,11,14-tetraazahexadecan-16-oato(3-)]gadolinium (OptiMARK).

These encapsulated chelate dendritic polymers are able to be administered in a variety of forms suitable for use as contrast agents. Because some of these encapsulated chelate dendritic polymers are crystalline solids, they can be administered orally or dissolved and administered as injectables, whether by intravenous injection, intramuscular, or intrapartioneal. Suitable excipients, buffers, diluents and other inert additives which assist in the stability of the administered encapsulated chelate dendritic polymer formulation as a pharmaceutical may be used. The various pharmaceutical forms may be used such as ampoules, tablets, capsules, solutions for injection, or other forms for the desired site in the animal body or ease of administration.

These encapsulated chelate dendritic polymers are expected to function as contrast agents because, for example, the chelate Magnevist™ is an FDA approved MRI contrast agent that is widely used and known to function as a contrast agent in an animal body such as humans and veterinary applications, as indicated by the listing of commercial contrast agents, and recent studies by Hisataka Kobayashi and Martin W. Brechbiel [Molecular Imaging 2(1), 1-10 (January 2003)]. These authors show that dendritic polymers can function as MRI agents when Gd is chelated on its surface. However, when the chelate is exclusively on the surface of a dendrimer the chelate is more exposed to the conditions of the body fluids, may exhibit unfavorable solubilities and biodistribution features and does not allow for the presentation of desirable targeting moieties or other favorable biodistribution functions.

In contrast to the known commercial imaging agents, the encapsulated chelate dendritic polymers may have increased stability of the product with a size control to avoid leakage out of the capillaries, and have less toxic effect possible because of reduced exposure of the ligand and metal and also still be eliminated through the kidneys. Thus the present encapsulated chelate dendritic polymers preferably have a size range of from about 1 to about 60 kD or about 1 nm to 4 nm for elimination through the kidneys, from 4 nm to 7 nm they can be eliminated through liver or bile, and when desired can be larger to 7 to 11 nm for imaging the liver and elimination in the bile.

The following examples further illustrate the invention but are not to be construed as a limitation on the scope of the invention. The lettered examples concern the preparation of comparative molecules; the numbered examples concern the preparation of products within the scope of this invention; and the Roman numerated examples show a use of some encapsulated chelate dendritic polymers of the invention.

### EXAMPLES

### Example 1

### PAMAM, EDA Core, Z OH (A) and NH₂ (B) with DTPA-Gd⁺³

Diethylenetriaminepentaacetic acid, gadolinium (III) dihydrgen salt (DTPA-Gd⁺³) is known commercially as Magnevist™ (by Schering AG). The structure of Magnevist™ has two free carboxylic acid groups. Magnevist™ was purchased from Aldrich (catalog #38,166-7). Using Magnevist™ as the chelate, it was encapsulated within a dendritic polymer of the poly(amidoamine) (PAMAM dendrimer class.

A. The PAMAM dendritic polymer possessed tris-hydroxymethyl groups on its surface as Z groups. The chelate is believed to be facilitated to the interior of the PAMAM due to the formation of an amine salt between the interior tertiary amines and the carboxylic acid groups of the chelate. Aqueous solutions of generation (G4 and G5 ethylenediamine (EDA) core, tris-OH surface PAMAM dendrimers were treated with a large excess of DTPA-Gd⁺³ as the chelate at room temperature (about 22° C.) for 48 hours. Then the mixture was subjected to dialysis extensively against water. Any free chelate should be removed after this procedure. Solvent was removed after the dialysis to give a fine, white solid as the product. The weight gains of the treated dendrimers suggest that a well defined complex between the dendrimer and the chelate are formed. The results are shown in Table 1 below. PAGE and MALDI-TOF analysis confirmed these results. Furthermore, the solubility of DTPA-Gd⁺³ was observed to be enhanced dramatically after the encapsulation procedure.

While not wishing to be bound by theory, we believe that this encapsulation presumably increases the rigidity of the dendrimer and enhances the three dimensional shape of the encapsulated chelate dendritic polymer forcing it to retain a more persistent, robust spherical structure compared to dendritic polymers not possessing such encapsulated chelates.

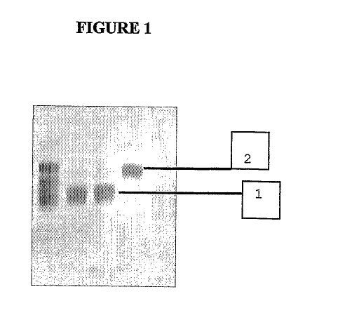

MALDI-MS spectrum also showed strong evidence for the formation of a dendrimer DTPA-Gd⁺³ complex. There are two to three peaks for each sample, including one matching the molar ratio calculated from the weight gain result. Since the encapsulation occurs inside the dendritic structure, PAGE showed almost no size change of the dendrimer after the encapsulation, respectively. See FIG. 1 for the G4-OH DTPA-Gd⁺³ shown as number 1 on the Figure and G5-OH DTPA-Gd⁺³ complex shown as number 2 on the Figure.

B. The dendritic polymer of PAMAM had primary amine groups on its surface as Z groups. Because DTPA-Gd⁺³ as the chelate contains two carboxylic acid groups in its structure, it can form amine salts both at the surface and in the interior of dendrimer. First a G4 PAMAM dendrimer was used to encapsulate the chelate. Following the standard procedure as in A above, the weight gain of dendrimer after the conjugation showed that about 32 chelate molecules are either encapsulated or form salts with each dendrimer molecule. MALDI-TOF gave a mass result that was consistent with this weight ratio. The generation 2 and 3 amine surfaced PAMAM dendrimers were used as hosts to conjugate with the chelate. The results are shown in Table 1.

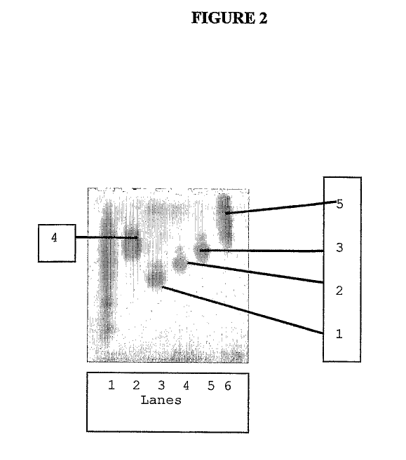

PAGE analysis of these DTPA-Gd⁺³ loaded conjugate compounds showed almost the same migration of the naked dendrimers (see FIG. 2). Apparently, that is because the surface functional groups of the conjugation products are amine, same as the dendrimer host molecules. However, the physical properties of the dendrimer chelate are different. Amine surface dendrimers are generally honey-like sticky materials; whereas the chelate dendrimers are fine, white solids. This must result because after the encapsulation, the rigidity of dendrimer increased drastically, making them fine solids.

Furthermore, in the presence of large excesses of DTPA-Gd⁺³, a coupling reagent was added to see if it was possible to achieve both encapsulation inside and coupling at the surface of amine surface dendrimer (Example 5 hereafter). Following the prior procedure, the weight gain showed there are 95 DTPA-Gd⁺³ per dendrimer, rather than 59 DTPA-Gd⁺³ per dendrimer without adding coupling reagents. Thus surface attachment also occurred of the chelate. PAGE showed the size of the product is substantially bigger than a non-chelated G4 dendrimer. (see FIG. 2, lane 2, #4). FIG. 2 shows at lane 3 a G2 (#1), at lane 4 a G3 (#2) and at lane 5 a G4 (#3) amine surface dendrimer with the chelate. Lane 6 (#5) is Example 5 where carbodiimide was added to the surface.

Table 1 below shows various Dendrimer PAMAM molecules with various generations and surfaces chelated with DTPA-Gd⁺³ as the chelate. In the Table below G=generation of the dendrimer; Z=the surface groups on the dendrimer and the number present; M=the chelate. All the samples of encapsulated chelate dendritic polymers were fine, white powders, and water soluble.

TABLE 1

| | | | | Dendrimer | Avg. | Avg. | | |
|---|---|---|---|---|---|---|---|---|
| | | | Interior | (D) | D:M | D:M | | Diameter |
| | Z | Z | Tertiary | Molecular | Molar | Wt. | MALDI | PAGE |
| G | Group | No. | Amine^{a} | Wt. | Ratio | Ratio^{b} | Peaks^{d} | nm |
| 4 | OH | | 62 | 18131 | 1:32 | 1:0.95 | 34485 | 4.2 |
| | | | | | | | (1:30) | |
| 5 | OH | | 126 | 36638 | 1:82 | 1:1.23 | 78971 | 5.1 |
| | | | | | | | (1:77) | |
| 4 | NH₂ | 64 | 62 | 14215 | 1:59 | 1:2.25^{C} | 46617 | 4.5 |
| | | | | | | | (1:58) | |
| 3 | NH₂ | 32 | 30 | 6909 | 1:38 | 1:3.07^{C} | 25851 | 3.6 |
| | | | | | | | (1:35) | |
| 2 | NH₂ | 16 | 14 | 3256 | 1:20 | 1:3.39^{C} | —^{e} | 2.9 |
| 4 | Pyrrolidone | | 62 | 22285 | 1:36 | 1:0.90 | 48000 | |
| | | | | | | | (1:46) | |
| 4 | OH | | 62 | 14277 | 1:32 | 1.1.25 | 36169 | |
| | (EA) | | | | | | (1:39) | |

^{a}= the tertiary amine inside the dendritic polymer structure is believed to be the bonding sites of the guest chelate.
^{b}= the ratio is based on the weight gain of the dendritic polymer after encapsulation of the chelate and after extensive (exhaustive) dialysis.
^{C}= the amine surface dendrimers take more chelate molecules to bond on the surface.
^{d}= there were 2 to 3 MALTI-TOF mass peaks run for each sample. The one entered in the Table is the closest to the weight gain calculation.
g = this data point was only the peak of the G2 dendrimer.

This data shows that when the surface groups, Z, are selected that do not bond with the chelate, then the diameter size of the encapsulated dendritic polymer is not effected as the chelate in encapsulated and the loading of the chelate is related to the interior amines present. However, when the surface groups, Z, can bond with the chelate, then the diameter size of the encapsulated chelate dendritic polymer is effected and becomes larger, and the total number of chelate groups present increases beyond that shown when the chelated groups are encapsulated without surface attachment, indicating further bonding of chelate groups on the surface. Since extensive dialysis was done to these chelate groups, whether inside the dendrimer or on the surface, it was shown that they were not easily disassociated from the dendrimer.

### Example 2

### Dendritic Polymer=PAMAM, G2, EDA Core, Z NH₂; Chelate=DTPA-Gd⁺³

A methanol solution of 0.5 g of a G2, EDA core, NH₂ surface PAMAM dendrimer was dried under vacuum to give 112 mg (0.0344 mmol) of dry dendrimer. Water (7 mL) was added to dissolve the dendrimer. Then 848 mg (1.548 mmol) of chelate was added to the solution. The mixture was stirred at room temperature (ca. 22° C.) for 48 hours. Undissolved solid was filtered off. Dialysis of the solution against water was done using 1,000 cut-off cellulose membrane for 4.5 hours with several water changes. Solvent water was removed by rotary-evaporation. The residue was put on high vacuum to yield 492 mg of a white solid (weight gain 380 mg).

### Example 3

### Dendritic Polymer=PAMAM, G3, EDA Core, Z NH₂; Chelate=DTPA-Gd⁺³

A methanol solution of 0.5 g of a G3, EDA core, NH₂ surface PAMAM dendrimer was dried under vacuum to give 109 mg (0.0158 mmol) of dry dendrimer. Water (7 mL) was added to dissolve the dendrimer. Then 804 mg (1.467 mmol) of chelate was added to the solution. The mixture was stirred at room temperature (ca. 22° C.) for 48 hours. Undissolved solid was filtered off. Dialysis of the solution against water was done using 1,000 cut-off cellulose membrane for 4.5 hours with several water changes. Solvent water was removed by rotary-evaporation. The residue was put on high vacuum to yield 444 mg of a white solid (weight gain 335 mg), MALDI-TOF of 11804,25851.

### Example 4

### Dendritic Polymer=PAMAM, G4, EDA Core, Z NH₂; Chelate=DTPA-Gd⁺³

A methanol solution of 2.0 g of a G4, EDA core, NH₂ surface PAMAM dendrimer was dried under vacuum to give 226 mg (0.0159 mmol) of dry dendrimer. Water (7 mL) was added to dissolve the dendrimer. Then 543 mg (0.99 mmol) of chelate was added to the solution. The mixture was stirred at room temperature (ca. 22° C.) for 48 hours. Undissolved solid was filtered off. Dialysis of the solution against water was done using 1,000 cut-off cellulose membrane for 2.5 hours with several water changes. Solvent water was removed by rotary-evaporation. The residue was put on high vacuum to yield 730 mg of a white solid (weight gain 510 mg), MALDI-TOF of 20337, 46617.

### Example 5

### Dendritic Polymer=PAMAM, G4, EDA Core, Z NH₂; Chelate=DTPA-Gd⁺³; Carbodiimide Modified

A methanol solution of 2.0 g of a G4, EDA core, NH₂ surface PAMAM dendrimer was dried under vacuum to give 226 mg (0.0159 mmol) of dry dendrimer. Water (6.5 mL) was added to dissolve the dendrimer. Then 1,100 mg (2.007 mmol) of chelate was added to the solution. The mixture was stirred at room temperature (ca. 22° C.) for 48 hours. There was undissolved solid in the mixture. Then 1.5 g (7.82 mmol) of 1-[3-(dimethylamino)propyl]-3-ethylcarbodiimide hydrochloride was added to the reaction mixture. The solution became slightly yellow with no undissolved solid present. The reaction was stirred for 24 hours at room temperature. Dialysis of the solution against water was done using 1,000 cut-off cellulose membrane for 4.5 hours with several water changes. Solvent water was removed by rotary-evaporation. The residue was put on high vacuum to yield 1.279 g of a white solid (weight gain 1.053 g), MALDI-TOF of 23210, 51987.

### Example 6

### Dendritic Polymer=PAMAM, G4, EDA Core, Z OH(Tris); Chelate DTPA-Gd⁺³

To a water solution of 2.0 g of a G4, EDA core, OH(tris) surface PAMAM dendrimer (10.1%, 214 mg dry dendrimer, 0.0118 mmol) was added 390 mg (0.712 mmol) of chelate. The mixture was stirred at room temperature (ca. 22° C.) for 48 hours. Dialysis of the solution against water was done using 3,500 cut-off cellulose membranes for 2.5 hours with several water changes. Solvent water was removed by rotary-evaporation. The residue was put on high vacuum to yield 632 mg of a white solid (weight gain 418 mg), MALDI-TOF of 34485, 53351.

### Example 7

### Dendritic Polymer=PAMAM, G5, EDA Core, Z OH(Tris); Chelate=DTPA-Gd⁺³

To a water solution of 2.0 g of a G5, EDA core, OH(tris) surface PAMAM dendrimer (10%, 200 mg dry dendrimer, 0.00546 mmol) was added 381 mg (0.695 mmol) of chelate. The mixture was stirred at room temperature (ca. 22° C.) for 48 hours. Dialysis of the solution against water was done using 3,500 cut-off cellulose membranes for 4.5 hours with several water changes. Solvent water was removed by rotary-evaporation. The residue was put on high vacuum to yield 446 mg of a white solid (weight gain 246 mg), MALDI-TOF of 38813, 78971.

### Example 8

### Dendritic Polymer=PAMAM, G4, EDA Core, Z OH(Tris); Chelate=DTPA-Gd⁺³; Dialysis Against 1×PBS Buffer (Similar to Human Serum)

To a water solution of 2.0 g of a G4, EDA core, OH(tris) surface PAMAM dendrimer (10.1%, 200 mg dry dendrimer, 0.0118 mmol) was added 390 mg (0.712 mmol) of chelate. The mixture was stirred at room temperature (ca. 22° C.) for 48 hours. Dialysis of the solution against 1×PBS (phosphate buffered saline solution) was done using 1,000 cut-off cellulose membrane for 2.5 hours with several buffer changes, followed by dialysis against deionized water for 1.5 hours. Solvent water was removed by rotary-evaporation. The residue was put on high vacuum to yield 345 mg of a white solid (weight gain 145 mg); with about 24 chelate per dendrimer (molar ratio).

### Example 9

### Dendritic Polymer=PAMAM, G4, EDA Core, Z Pyrrolidone; Chelate=DTPA-Gd⁺³

A methanol solution of 2.0 g of a G4, EDA core, pyrrolidone surface PAMAM dendrimer was dried under vacuum to give 200 mg (0.00898 mmol) of dry dendrimer. Water (7 mL) was added to dissolve the dendrimer. Then 305 mg (0.556 mmol) of chelate was added to the solution. The mixture was stirred at room temperature (ca. 22° C.) for 48 hours. Dialysis of the solution against water was done using 1,000 cut-off cellulose membrane for 4.0 hours with several water changes. Solvent water was removed by rotary-evaporation. The residue was put on high vacuum to yield 379 mg of a white solid (weight gain 179 mg), MALDI-TOF of 52484; with about 36 chelate per dendrimer (molar ratio).

### Example 10

### Dendritic Polymer=PAMAM, G4, EDA Core, Z Monohydroxyl; Chelate=DTPA-Gd⁺³

A methanol solution of 2.0 g of a G4, EDA core, monohydroxyl surface PAMAM dendrimer was dried under vacuum to give 220 mg (0.0154 mmol) of dry dendrimer. Water (7 mL) was added to dissolve the dendrimer. Then 538 mg (0.982 mmol) of chelate was added to the solution. The mixture was stirred at room temperature (ca. 22° C.) for 48 hours. Dialysis of the solution against water was done using 1000 cut-off cellulose membrane for 4.0 hours with several water changes. Solvent water was removed by rotary-evaporation. The residue was put on high vacuum to yield 494 mg of a white solid (weight gain 274 mg), MALDI-TOF of 36169; with about 32 chelate per dendrimer (molar ratio).

### Example 11

### Dendritic Polymer=PAMAM, G4, EDA Core, Z OH(Tris); Chelate=DOTP-Gd⁺³

To 33 mg (0.00182 mmol) of a G4, EDA core, OH(tris) surface PAMAM dendrimer was added 2.0 mL of deionized water. To this solution was added 50 mg (0.0577 mmol) of chelate. The mixture was stirred at room temperature (ca. 22° C.) for 2 days. Dialysis of the solution against deionized water was done using 1,000 cut-off cellulose membrane for 4.0 hours with several water changes. Solvent water was removed by rotary-evaporation. The residue was put on high vacuum to yield 75 mg of a white solid (weight gain 42 mg); with about 27 chelate per dendrimer (molar ratio).

### Example 12

### Dendritic Polymer=PAMAM, G4, EDA Core, Z OH(Tris); Chelate=DOTA-Gd⁺³

To 36 mg (0.00198 mmol) of a G4, EDA core, OH(tris) surface PAMAM dendrimer was added 2.0 mL of deionized water. To this solution was added 50 mg (0.0806 mmol) of chelate. The mixture was stirred at room temperature (ca. 22° C.) for 2 days. Dialysis of the solution against deionized water was done using 1,000 cut-off cellulose membrane for 4.0 hours with several water changes. Solvent water was removed by rotary-evaporation. The residue was put on high vacuum to yield 52 mg of a white solid (weight gain 16 mg); with about 15 chelate per dendrimer (molar ratio).

### Example 13

### Dendritic Polymer=PAMAM, G4, EDA Core, Z NH₂ with Polyethylene Glycol Modification (PEG); Chelate=DTPA-Gd⁺³

To a solution of 14 imol of the PAMAM dendrimer in 10 mL of dimethyl sulfoxide was added 0.9 mmol of M-PEG 4-nitrophenyl carbonate. The solution was stirred for 5 days at room temperature (ca. 22° C.). The solution was diluted with distilled water and then dialyzed with a dialysis bag (cut off of 12,000-14,000) against distilled water for 24 hours. The crude product was lyophized and then purified either by Sephadex G-75 column (Pharmacia, 4 cm-45 cm) using water as the eluent in the case in which M-PEG(2000) was used or by a Sephadex LH-20 column using methanol as the eluent in the case in which M-PEG(550) was used.

A methanol solution of 2.0 g of a G4, EDA core, pegalated surface PAMAM dendrimer was dried under vacuum to give 205 mg (0.00415 mmol) of dry dendrimer. Water (7 mL) was added to dissolve the dendrimer. Then 141 mg (0.2572 mmol) of chelate was added to the solution. The mixture was stirred at room temperature (ca. 22° C.) for 48 hours. Dialysis of the solution against water was done using 1,000 cut-off cellulose membrane for 5 hours with several water changes. Solvent water was removed by rotary-evaporation. The residue was put on high vacuum to yield 287 mg of a white solid (weight gain 82 mg), with a dendrimer:chelate of about 1:36.1 (molar ratio),

### Example 14

### Dendritic Polymer=PAMAM, G3, EDA Core, Z NH₂ with Polyethylene Glycol Modification (PEG); Chelate=DTPA-Gd⁺³

To a solution of 14 imol of the PAMAM dendrimer in 10 mL of dimethyl sulfoxide was added 0.9 mmol of M-PEG 4-nitrophenyl carbonate. The solution was stirred for 5 days at room temperature (ca. 22° C.). The solution was diluted with distilled water and then dialyzed with a dialysis bag (cut off of 12,000-14,000) against distilled water for 24 hours. The crude product was lyophized and then purified either by Sephadex G-75 column (Pharmacia, 4 cm-45 cm) using water as the eluent in the case in which M-PEG(2000) was used or by a Sephadex LH-20 column using methanol as the eluent in the case in which M-PEG(550) was used.

A methanol solution of 2.0 g of a G3, EDA core, pegalated surface PAMAM dendrimer was dried under vacuum to give 208 mg (0.00849 mmol) of dry dendrimer. Water (7 mL) was added to dissolve the dendrimer. Then 140 mg (0.255 mmol) of chelate was added to the solution. The mixture was stirred at room temperature (ca. 22° C.) for 48 hours. Dialysis of the solution against water was done using 1,000 cut-off cellulose membrane for 5 hours with several water changes. Solvent water was removed by rotary-evaporation. The residue was put on high vacuum to yield 280 mg of a white solid (weight gain 72 mg); with a dendrimer:chelate of about 1:15.5 (molar ratio).

### Example 15

### Dendritic Polymer=PAMAM, G2, EDA Core, Z NH₂ with Polyethylene Glycol Modification (PEG); Chelate=DTPA-Gd⁺³

To a solution of 14 imol of the PAMAM dendrimer in 10 mL of dimethyl sulfoxide was added 0.9 mmol of M-PEG 4-nitrophenyl carbonate. The solution was stirred for 5 days at room temperature (ca. 22° C.). The solution was diluted with distilled water and then dialyzed with a dialysis bag (cut off of 12,000-14,000) against distilled water for 24 hours. The crude product was lyophized and then purified either by Sephadex G-75 column (Pharmacia, 4 cm-45 cm) using water as the eluent in the case in which M-PEG(2000) was used or by a Sephadex LH-20 column using methanol as the eluent in the case in which M-PEG(550) was used.

A methanol solution of 2.0 g of a G2, EDA core, pegalated surface PAMAM dendrimer was dried under vacuum to give 208 mg (0.0173 mmol) of dry dendrimer. Water (7 mL) was added to dissolve the dendrimer. Then 132 mg (0.242 mmol) of chelate was added to the solution. The mixture was stirred at room temperature (ca. 22° C.) for 48 hours. Dialysis of the solution against water was done using 1,000 cut-off cellulose membrane for 5 hours with several water changes. Solvent water was removed by rotary-evaporation. The residue was put on high vacuum to yield 277 mg of a white solid (weight gain 69 mg); with a dendrimer:chelate of about 1:7.3 (molar ratio).

### Example 16

### Dendritic Polymer=PAMAM, G2, EDA Core, Z OH(Tris); Chelate=DTPA-Gd⁺³

To a water solution of 2.0 g of a G2, EDA core, OH(tris) surface PAMAM dendrimer (10.1%, 200 mg dry dendrimer, 0.0472 mmol) was added 362 mg (0.661 mmol) of chelate. The mixture was stirred at room temperature (ca. 22° C.) for 48 hours. Dialysis of the solution against water was done using 1,000 cut-off cellulose membrane for 5 hours with several water changes. Solvent water was removed by rotary-evaporation. The residue was put on high vacuum to yield 363 mg of a white solid (weight gain 163 mg); with a dendrimer:chelate of about 1:6.3 (molar ratio).

### Example 17

### Dendritic Polymer=PAMAM, G3, EDA Core, Z OH(Tris); Chelate=DTPA-Gd⁺³

To a water solution of 2.0 g of a G3, EDA core, OH(tris) surface PAMAM dendrimer (10.1%, 200 mg dry dendrimer, 0.0226 mmol) was added 548 mg (0.677 mmol) of chelate. The mixture was stirred at room temperature (ca. 22° C.) for 48 hours. Dialysis of the solution against water was done using 1,000 cut-off cellulose membrane for 5 hours with several water changes. Solvent water was removed by rotary-evaporation. The residue was put on high vacuum to yield 379 mg of a white solid (weight gain 179 mg); with a dendrimer:chelate of about 1:14.5 (molar ratio).

### Example I

### Relaxivity Studies

In accord with standard practices, in the following table was prepared from the samples from the indicated examples using 20 MHz, 10 mL of phosphate buffered saline (PBS) to suspend or dissolve the sample, 200 μL of solution put in the NMR tube, chelate was DTPA-Gd⁺³. [For an example of the procedure see Nuclear magnetic Resonance Imaging Basic Principles by Stuart W. Young, MD, (1984), pub. Raven Press.]

TABLE 2

| | | | Example | Amount | |
|---|---|---|---|---|---|
| Example | Chelate % | Average | quantity | used | |
| No. | (w/w) | MW | (mg) | (mg) | [Gd(III)]M |
| 6 | 48.8 | 35,667 | 260 | 20.8 | 0.001854 |
| 7 | 53.5 | 78,971 | 260 | 20.6 | 0.002013 |
| 4 | 69.9 | 46,547 | 300 | 21.4 | 0.002732 |
| 5 | 79.0 | 66,275 | 300 | 20.6 | 0.002972 |
| 2 | 77.2 | 14,216 | 300 | 22.3 | 0.003144 |
| 3 | 75.4 | 25,851 | 265 | 19.8 | 0.002726 |
| 9 | 47.2 | 43,013 | 250 | 25.4 | 0.002189 |
| 10 | 55.5 | 31,813 | 300 | 28.1 | 0.002848 |
| Control | 0 | N/A | N/A | N/A | 0 |

| Example | T1 | R1 | r1 1/ | R2 | r2 | T2 |
|---|---|---|---|---|---|---|
| No. | (msec) | 1/(sec) | (sec mM) | 1/(sec) | (sec mM) | (msec) |
| 6 | 105.0 | 9.5238 | 4.9409 | 11.9190 | 6.0836 | 87.9 |
| 7 | 105.0 | 9.5238 | 4.5506 | 11.3636 | 5.3272 | 87.1 |
| 4 | 79.4 | 12.5945 | 4.4768 | 15.4560 | 5.4229 | 66.9 |
| 5 | 78.0 | 12.8205 | 4.1910 | 17.2414 | 5.5853 | 59.6 |
| 2 | 68.9 | 14.5138 | 4.5003 | 17.6056 | 5.3957 | 57.6 |
| 3 | 79.5 | 12.5786 | 4.4798 | 14.7059 | 5.1585 | 65.7 |
| 9 | 96.1 | 10.4058 | 4.5861 | 13.4590 | 5.8541 | 78.9 |
| 10 | 70.1 | 14.2653 | 4.8806 | 16.0000 | 5.3925 | 59.9 |
| Control | 2740.0 | 0.3650 | | 0.6418 | | 1543.0 |

R1=1T1; it is the longitudinal relaxation rate and has units of inverse seconds r1=(R1ₛ-R1₀)/[M]; r1 is the longitudinal relaxivity, R1ₛ is the longitudinal relaxation rate of the sample with paramagnetic agent of the indicated example, and R1₀ is the longitudinal relaxation rate of the buffer or sample without the paramagnetic agent. The units are inverse sec inverse mM. Substituting a 2 for the 1 gives the transverse values.

The results from the above Table 2 indicate that these examples provide, within experimental error, the same result as DTPA-Gd+3™ (3.7 to 4.8) without dendritic polymer. The\us the presence of the dendritic polymer does not impede the ability to use these encapsulated dendritic polymers as contrast agents. However, the present encapsulated chelate agents differ from the known contrast agents because they are of a controlled size that will not appreciably leak out of the capillaries. Thus the use of these present encapsulated chelate dendritic polymers as a blood pool agent is very likely.

### Comparative Examples

### Example A

### Dendritic Polymer=PAMAM, G4, EDA Core, Z OH(Tris); Ligand DTPA; No Metal Added

To a water solution of 2.0 g of a G4, EDA core, OH(tris) surface PAMAM dendrimer (10%, 200 mg dry dendrimer, 0.0114 mmol) was added 277 mg (0.706 mmol) of DTPA. The mixture was stirred at room temperature (ca. 22° C.) for 48 hours. Undissolved solid was filtered off. Dialysis of the solution against water was done using 1,000 cut-off cellulose membrane for 4.5 hours with several water changes. Solvent water was removed by rotary-evaporation. The residue was put on high vacuum to yield 333 mg of a white solid (weight gain 133 mg).

### Example B

### Dendritic Polymer=PAMAM, G4, EDA Core, Z OH(Tris); Ligand EDTA; Copper Cu Added

To a water solution of 2.013 g of a G4, EDA core, OH(tris) surface PAMAM dendrimer (10%, 203.3 mg dry dendrimer, 0.0112 mmol) was added 258 mg (0.695 mmol) of EDTA. The mixture was stirred at room temperature (ca. 22° C.) for 48 hours. The mixture was clear. Then 221.8 mg (1.34 mmol) of CuSO₄ was added. The solution became bright blue. The mixture was stirred for 24 hours. Dialysis of the solution against water was done using 1,000 cut-off cellulose membrane for 8 hours with several water changes. The rate of leaking of blue substance from the dendrimer compared to a control of EDTA and Cu+² is much slower. After 2 hours the control solution became clear, whereas the encapsulated dendrimer sample required 8 hours to become clear.

### Example C

### Dendritic Polymer=PAMAM, G4, EDA Core, Z OH(Tris); Ligand DOTA; No Metal Added

To a water solution of 0.5 g of a G4, EDA core, OH(tris) surface PAMAM dendrimer (10%, 50 mg dry dendrimer, 0.00276 mmol) was added 69.1 mg (0.171 mmol) of DOTA. The mixture was stirred at room temperature (ca. 22° C.) for 15 minutes. The solution became clear. The mixture was stirred at room temperature for 48 hours. Dialysis of the solution against water was done using 1,000 cut-off cellulose membrane for 4.5 hours with several water changes. Solvent water was removed by rotary-evaporation. The residue was put on high vacuum to yield 87 mg of a white solid (weight gain 37 mg); with a dendrimer to DOTA molar ratio of about 1:30.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

What is claimed is:

1. An encapsulated chelate dendritic polymer.

2. The encapsulated chelate dendritic polymer of claim 1 wherein the metal in the chelate is selected from the Periodic Table Groups VIIIA (Fe, Co, Ni, Ru, Rh, Pd, Os, Ir, Pt), IVB (Pb, Sn, Ge), IIIA (Sc, Y, lanthanides and actinides), IIIB (B, Al, Ga, In, Tl), IA (Li, Na, K, Rb, Cs, Fr), and IIA (Be, Mg, Ca, Sr, Ba, Ra).

3. The encapsulated chelate dendritic polymer of claim 2 wherein the metal is selected from gadolinium (Gd⁺³), iron (Fe⁺³), manganese (Mn⁺²) and (Mn⁺³), and chromium (Cr⁺³).

4. The encapsulated chelate dendritic polymer of claim 1 wherein the metal is lead, plutonium, iron, calcium, mercury, gold or other heavy metals or their ions.

5. The encapsulated chelate dendritic polymer of claim 1, 3 or 4 wherein the dendritic polymer is selected from polyamidoamine and poly(propyleneimine) dendrimers.

6. The encapsulated chelated dendritic polymer of claim 1, 3 or 4 wherein the chelating agent is selected from linear organic acids, macrocyclics, macrocyclic derivatives, kryptates, phosphines, thioalkyl, ethers, carboxylates, thioureas, phosphonic acids, methylenephosphonic acids, sulfonic acids, and macrocyclic polypeptides.

7. The encapsulated chelated dendritic polymer of claim 6 wherein the chelating agent is diethylenetriaminepentaacetic acid (DTPA), nitrilotriacetic acid (NTA), ethylenediaminetetraacetic acid (EDTA), hydroxyethylethylenediaminetriacetic acid (HEDTA), trans-1,2-diaminocyclohexanetetraacetic acid (CDTA), 1,4,7,10-tetraazacyclo-dodecane-1,4,7-triacetic acid (DO3A), 1,4,7,10-tetraazacyclododecanetetraacetic acid (DOTA), 1,4,7,10-tetraazacyclo-dodecane-1,4,7-triacetic acid (DO3A), 2-(p-isothiocyanatobenzyl)-6-methyl-diethylenetriaminepentaacetic acid (IB4M), hydroxypyridinone (HOPO) or TREN-1-methyl-3,2-hydroxypyridinone.

8. The encapsulated chelate dendritic polymer of claim 1 or 5 wherein the chelate is gadolinium diethylenetriaminepentaacetic acid (Gd DTPA).

9. The encapsulated chelate dendritic polymer of claim 5 wherein the dendritic polymer has as its surface groups hydroxyl, sulfhydryl groups.

10. The encapsulated chelate dendritic polymer of claim 1 wherein the dendritic polymer is a generation (G) 5, ethylenediaminetetraacetic acid (EDA) core, tris-OH surface PAMAM dendrimer.

11. The encapsulated chelate dendritic polymer of claim 1 wherein the dendritic polymer is a generation (G) 4, ethylenediaminetetraacetic acid (EDA) core, tris-OH surface PAMAM dendrimer.

12. The encapsulated chelate dendritic polymer of claim 1 which has associated with its dendritic polymer surface target directors, proteins, DNA, RNA (including single strands) or any other moieties that will assist in diagnosis, therapy or delivery of this encapsulated chelate dendritic polymer.

13. A pharmaceutically acceptable formulation of the encapsulated chelate dendritic polymer of claim 1 or 12 with at least one pharmaceutically acceptable diluent, excipient or carrier present.

14. A method for administering the formulation of claim 13 for use in an animal or plant as a contrast agent wherein the encapsulated chelate dendritic polymer is administered by injection, tablet, ampoule, powder, liquid or intravenous to the animal or plant.

15. An encapsulated ligand dendritic polymer.

16. The encapsulated ligand dendritic polymer of claim 15 wherein the ligand is selected from linear organic acids, macrocyclics, macrocyclic derivatives, kryptates, phosphines, thioalkyl, ethers, carboxylates, thioureas, phosphonic acids, methylenephosphonic acids, sulfonic acids, and macrocyclic polypeptides.

17. The encapsulated ligand dendritic polymer of claim 16 wherein the ligand is diethylenetriaminepentaacetic acid (DTPA), nitrilotriacetic acid (NTA), ethylenediaminetetraacetic acid (EDTA), hydroxyethylethylenediaminetriacetic acid (HEDTA), trans-1,2-diaminocyclohexanetetraacetic acid (CDTA), 1,4,7,10-tetraazacyclo-dodecane-1,4,7-triacetic acid (DO3A), or 1,4,7,10-tetraazacyclododecanetetraacetic acid (DOTA), 1,4,7,10-tetraazacyclo-dodecane-1,4,7-triacetic acid (DO3A), 2-(p-isothiocyanatobenzyl)-6-methyl-diethylenetriaminepentaacetic acid (IB4M), hydroxypyridinone (HOPO) or TREN-1-methyl-3,2-hydroxypyridinone.

18. The encapsulated ligand of claim 15 or 16 wherein the dendritic polymer is selected from polyamidoamine and poly(propyleneimine) dendrimers.

19. A pharmaceutically acceptable formulation of the encapsulated ligand dendritic polymer of claim 15 with at least one pharmaceutically acceptable diluent, excipient or carrier present.

20. A method for administering the formulation of claim 15 for use in an animal or plant as a scavenger agent or for chelant therapy wherein the encapsulated ligand dendritic polymer is administered by injection, tablet, ampoule, powder, liquid or intravenous to the animal or plant.
